(19) United States
(12) Patent Application Publication
(10) Pub. No.: US 2014/0102452 A1
(43) Pub. Date: Apr. 17, 2014

(54) Tube in tube breathing circuits

(71) Applicant: Martin E. Forrester, Trenton (CA)

(72) Inventor: Martin E. Forrester, Trenton (CA)

(73) Assignee: SCHAUENBURG HOSE TECHNOLOGY GMBH

(21) Appl. No.: 13/998,898

(22) Filed: Dec. 19, 2013

Related U.S. Application Data
(63) Continuation-in-part of application No. 13/986,465, filed on May 6, 2013.
Application No. 13/986,465 is a continuation of application No. 12/799,263, filed on Apr. 21, 2010, now Pat. No. 8,453,681.
Application No. 12/799,263 is a continuation-in-part of application No. 12/354,291, filed on Jan. 15, 2009, now abandoned.

(51) Int. Cl. A61M 16/08

(52) U.S. Cl. CPC A61M 16/0875
USPC 128/204.18

(57) ABSTRACT

Similar lengths of corrugated stretch hose are components used to form a tube-in-tube breathing circuit assembly having a smaller diameter inner hose that extends loosely through a larger diameter outer hose. Overlying end regions of the inner and outer hoses are joined by connectors that communicate separately with each of the inner and outer hoses near opposite ends of the assembly. Exhaled breath from a patient that travels through the outer hose warms breathing gas supplied to the patient through the inner hose. In one form of preferred practice, the inner hose draws taut regardless of the extent to which the breathing circuit assembly may be extended, retracted and bent, which minimizes resistance to flow through each of the two hoses.

### CROSS-REFERENCE TO RELATED PATENT AND APPLICATIONS

This application is a continuation-in-part of application Ser. No. 13/986,465 filed May 6, 2013 entitled METHOD OF HOSE MANUFACTURE (Atty's Dkt. No. 6-459) which was filed as a continuation of application Ser. No. 12/779,263 filed Apr. 21, 2010 entitled FLEXIBLE, STRETCHABLE, CRUSH RESISTANT HOSE WELL SUITED FOR MEDICAL APPLICATIONS (Atty's Dkt. No. 6-451) which was filed as a continuation-in-part of application Ser. No. 12/354,291 filed Jan. 15, 2009 (now abandoned).

Application Ser. No. 12/779,263 claimed the benefit of the filing date of a provisional application Ser. No. 61/335,023 filed Dec. 30, 2009 entitled FLEXIBLE HOSE FOR MEDICAL APPLICATIONS (Atty's Dkt. No. 6-449).

Application Ser. No. 12/779,263 issued Jun. 4, 2013 as U.S. Pat. No. 8,453,861.

The disclosures of the above-listed patent and of all of the applications listed above are incorporated herein by reference.

### FIELD OF THE INVENTION

The present invention relates to a flexible, extensible and retractable assembly of two corrugated hoses for medical use—with the assembly including a smaller diameter inner hose that extends loosely through a larger diameter outer hose, and having end connectors that rigidly join each of the two sets of overlying end regions of the outer and inner hoses near opposite end regions of the assembly. Each of the end connectors provides separate paths of communication with the outer and inner hoses.

Hose assemblies of this type are often called “tube-in-tube breathing circuits,” and typically are used to duct exhaled breath away from a patient through the outer hose, while breathing gas is delivered to the patient through the inner hose. Warm exhaled breath moving through the larger diameter outer hose typically is used to heat such breathing gas as is supplied to the patient through the smaller diameter inner hose.

### BACKGROUND

Tube-in-tube breathing circuit assemblies that employ corrugated extensible hoses of differing diameter arranged one within another are known to extend and retract in an awkward fashion due primarily to conflicting extension and retraction characteristics of the outer and inner hoses. Stated in another way, because the outer and inner hoses of breathing circuit assemblies are separately formed from materials that may differ in composition, wall thickness, stiffness and the like, it is not unusual for the outer and inner hoses of a tube-in-tube breathing circuit assembly to resist or to essentially fight with each other when force is applied to cause the assemblies to extend, to retract, and/or to bend.

The extension and retraction characteristics of the larger mass, larger diameter outer hose of a tube-in-tube breathing circuit assembly commonly dominates the less predominant, less powerful extension and retraction characteristics of the smaller mass, smaller diameter inner hose. Stated in another way, how a breathing circuit assembly behaves during extension and retraction is usually governed and controlled primarily by how the outer hose of the assembly tends to behave during extension and retraction. While the extension and retraction behavior of the smaller diameter inner hose may control how the inner hose behaves during extension and retraction of the assembly, inner hose behavior seldom does little to affect the extension and retraction behavior of the assembly itself.

Sometimes differences in the extension and retraction characteristics of the outer and inner hoses of a breathing circuit assembly can diminish or detrimentally affect the performance of a tube-in-tube breathing circuit assembly. If, for example, the outer hose of a breathing circuit assembly is relatively easy to retract, but the inner hose of the assembly is stiff and therefore resists retraction, the domination of the inner hose by the outer hose may cause the assembly to be relatively easy to retract, but the stiffness of the inner hose may cause the inner hose to attempt to maintain its extended length by assuming a configuration that riggles from side to side, or (more correctly, in a three-dimensional world) takes on a spiral shape that hugs the interior surface of the outer hose (as closely as the corrugated inner hose can hug the interior of the corrugated outer hose) as the inner hose spirals along and through the interior of the outer hose. The wavey or spiralling configuration of the inner hose presents an increased resistance to flow not only to gas that is ducted through the inner hose, but also (and probably even more prominently) to the flow of gas that is ducted along the tortuous open path that is left open to such flow between the outer and inner hoses.

Some known efforts at providing tube-in-tube breathing circuit assemblies have utilized outer hoses that are selected from among corrugated stretch hoses that have a propensity to retain a configuration to which they have been extended, retracted and/or bent. Tube-in-tube assemblies that utilize such outer hoses nonetheless frequently have the problem referred to just above in that their inner hoses take on a wavey or spiral configuration when attempts are made to retract or shorten the assemblies, which causes unwanted increases to occur in flow resistance to the gases being ducted through the outer and inner hoses.

In some forms of preferred practice, the present invention takes advantage of such inventions as are disclosed 1) in U.S. Pat. No. 8,453,681 which issued Jun. 14, 2013, from application Ser. No. 12/779,263 filed Apr. 21, 2010, entitled FLEXIBLE, STRETCHABLE, CRUSH RESISTANT HOSE WELL SUITED FOR MEDICAL APPLICATIONS (Atty's Docket No. 6-451), and 2) in a continuation application thereof, Ser. No. 13/986,465 filed May 6, 2013, entitled METHOD OF HOSE MANUFACTURE (Atty's Docket No. 6-459). These referenced documents are referred to later herein as the “Hose Annealing Patents,” the disclosures of which documents are incorporated herein by reference.

The referenced Hose Annealing Patents disclose methods by which the properties of helically wound, accordion-like hoses formed from thermoplastic material can be stress relieved, can have their properties advantageously enhanced, and can have their enhanced properties “set” so as to be permanently retained by the “memory” of the stress-relieved hoses.

One of the teachings of the referenced Hose Annealing Patents is that, by annealing lengths of corrugated stretch hose formed from thermoplastic materials while the hose lengths are being axially compressed, it is possible to reset the “memory” that controls the length to which a newly formed hose will seek to return when it is stretched or otherwise caused to extend. For example, if a corrugated stretch hose formed from thermoplastic material is axially compressed to assume a minimum length, and, while axially compressed, is annealed (i.e., is heated to an elevated temperature selected to relieve such stress energy as may have become stored in the “memory” of the hose due to production techniques and the like, and is then slowly cooled to ambient temperature to complete the annealing procedure), the newly annealed hose now has a “reset memory” or a “revised normal state” or a “new remembered configuration” of minimal axial length to which the stress-relieved hose seeks to return when the hose is stretched or otherwise forcefully extended.

The term “reverse set” is a term of art that has come to be associated with a length of corrugated stretch hose formed from thermoplastic material that has been annealed while being axially compressed to its minimum axial length to cause its memory to be “reset” as just described. What is referred to by use of the term “reverse set” herein is the tendency of a length of hose formed from thermoplastic material (that has been annealed while being axially compressed—as is disclosed in the referenced Hose Annealing Patents) to return to a newly remembered condition of minimum axial length when force that stretches or causes the hose length to extend is no longer being applied to the length of hose.

### SUMMARY

A challenge addressed by some embodiments of the present invention is to provide tube-in-tube breathing circuit assemblies that employ extensible-retractable outer and inner hoses that have different extension and retraction characteristics which are used advantageously.

A further challenge addressed by some embodiments of the present invention is to provide tube-in-tube breathing circuit assemblies that extend, retract and bend in a manner that maintains efficient flows of gas through the outer and inner hoses of the assemblies regardless of the extent to which the assembly may be extended, retracted and/or bent during use.

Yet another challenge addressed by some embodiments of the invention is to provide tube-in-tube breathing circuit assemblies wherein the inner hose continues to draw taut regardless of the extent to which the assemblies may be extended, retracted and/or bent during use—which tensioning of the inner hose causes it to maintain a configuration that is as linear and short as possible, thereby minimizing the resistance to flow that is presented by both of the outer and inner hoses to gas flowing through the interiors of each of the outer and inner hoses regardless of the extent to which the assembly may be extended, retracted and bent during use of the assembly.

In some embodiments, a tube-in-tube breathing circuit assembly is provided that includes an inner hose of relatively small diameter that has been “reverse set” by means of an annealing process, and an outer hose that has not been reverse set.

In some embodiments, a tube-in-tube breathing circuit assembly is provided that includes an inner hose which has been “reverse set” and therefore tends to remain taut, and an outer hose selected from hoses that tend to retain the extended, retracted and bent configurations to which the outer hose has been extended, retracted and/or bent, thereby providing breathing circuit assemblies that tend to retain such extended, retracted and/or bent configurations to which the assemblies are extended, retracted and bent.

In some embodiments, a corrugated inner hose is “reverse set” to cause it to draw taut inside a larger diameter corrugated outer hose whenever and to whatever extent the outer hose is extended, retracted or bent, and thereby providing a tube-in-tube breathing circuit assembly that minimizes such flow resistance as is encountered by gas ducted through the outer and inner hoses.

In some forms of preferred practice, tube-in-tube breathing circuit assemblies are advantageously formed that each employ an inner hose that has a tendency to retract to a minimal length, and a dominant outer hose that has a tendency to retain whatever retracted, extended and bent configuration to which the assemblies are retraced, extended and bent—whereby the resulting breathing circuit assemblies having a tendency to remain in whatever retracted, extended and bent configurations to which the assemblies are positioned, while the inner hose tends to remain taut so it presents a minimum of flow resistance to exhaled breath of a patient ducted through the outer hose, while also providing a minimum of flow resistance to breathing gas ducted to the patient through the inner hose.

### BRIEF DESCRIPTION OF THE DRAWINGS

These and other features, and a fuller understanding of the invention may be had by referring to the following description, taken in conjunction with the accompanying claims and drawings, wherein:

FIG. 1 is a perspective view of a tube-in-tube breathing circuit assembly incorporating features of the present invention, with the assembly shown in an axially retracted attitude or form;

FIG. 2 is a perspective view, on a somewhat smaller scale, of substantially the same tube-in-tube breathing circuit assembly, with the assembly shown in an axially extended or stretched attitude or form;

FIG. 3 is a cross-sectional view of substantially the same tube-in-tube breathing circuit assembly depicted in FIGS. 1 and 2, with central regions of outer and inner hoses of the assembly foreshortened so that features of conventional end connectors of the assembly can be depicted with greater attention to detail; and,

FIG. 4 is a cross-sectional view of a center region of substantially the same tube-in-tube breathing circuit assembly shown in FIGS. 1-3, with the view also showing a preferred manner in which center regions of the outer and inner hoses behave somewhat differently when the assembly is bent.

### DESCRIPTION

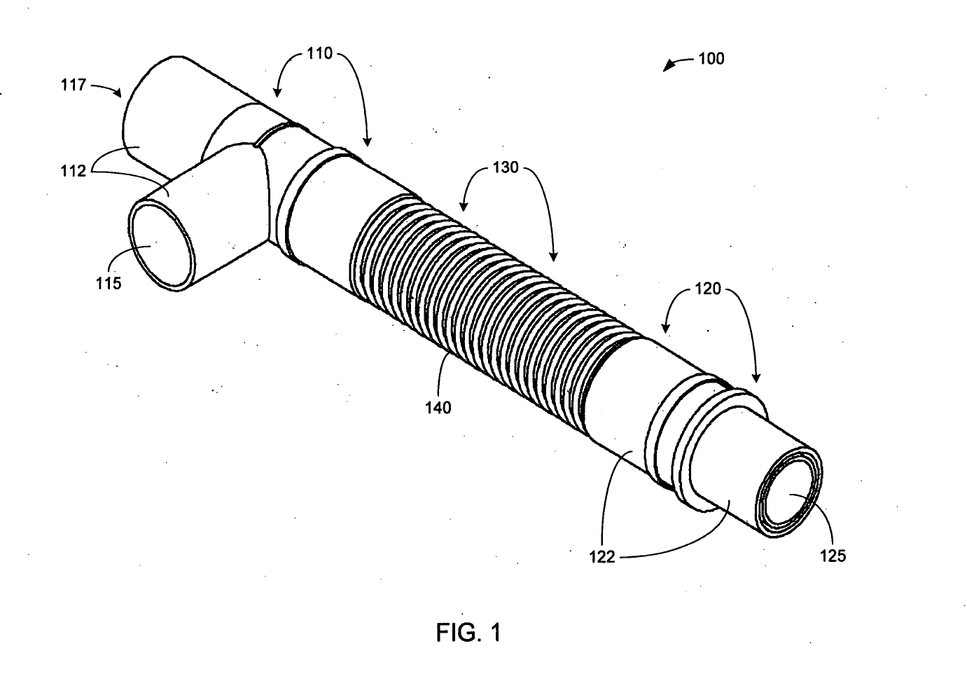

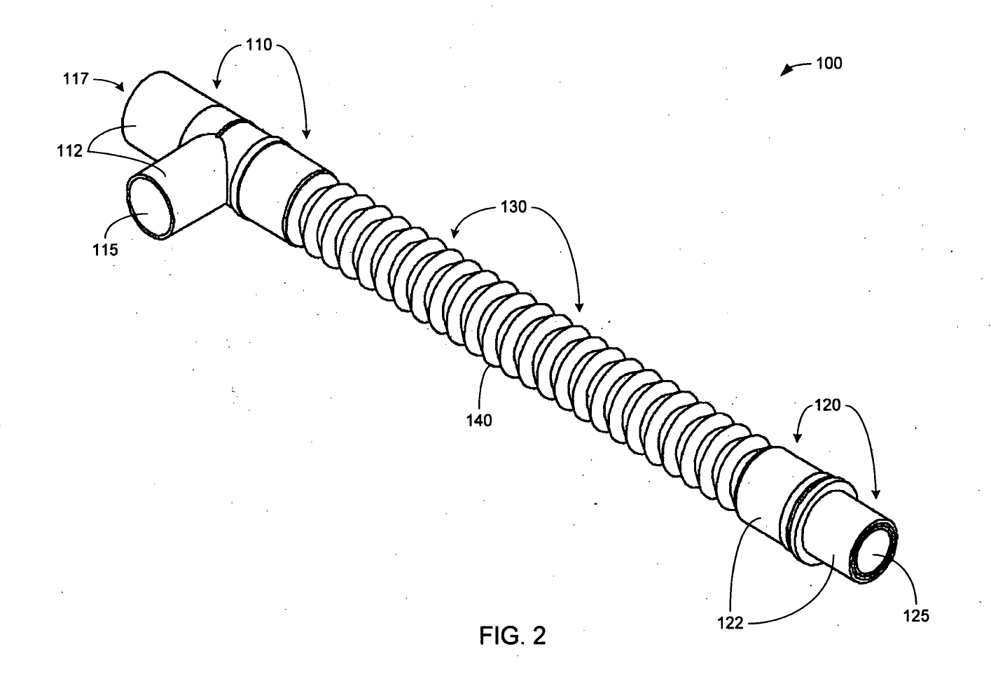

Referring to FIGS. 1 and 2, a tube-in-tube breathing circuit assembly embodying features of the present invention is indicated generally by the numeral 100. In FIGS. 1 and 2, first and second end regions of the assembly 100 are indicated generally by the numerals 110, 120, respectively, and a central region of the assembly 100 is indicated generally by the numeral 130. The first and second end regions 110, 120 are primarily defined by first and second end connector assemblies indicated by the numerals 112, 122, respectively. The central region 130 extends from the first end connector assembly 112 to the second end connector assembly 122.

In FIG. 1, the breathing circuit assembly 100 is shown in an axially retracted attitude or form—which means that the central region 130 is shown in substantially a minimum axially retracted length. In FIG. 2, the breathing circuit assembly 100 is shown in an axially extended attitude or form—which means that the central region 130 is shown in substantially a maximally extended length.

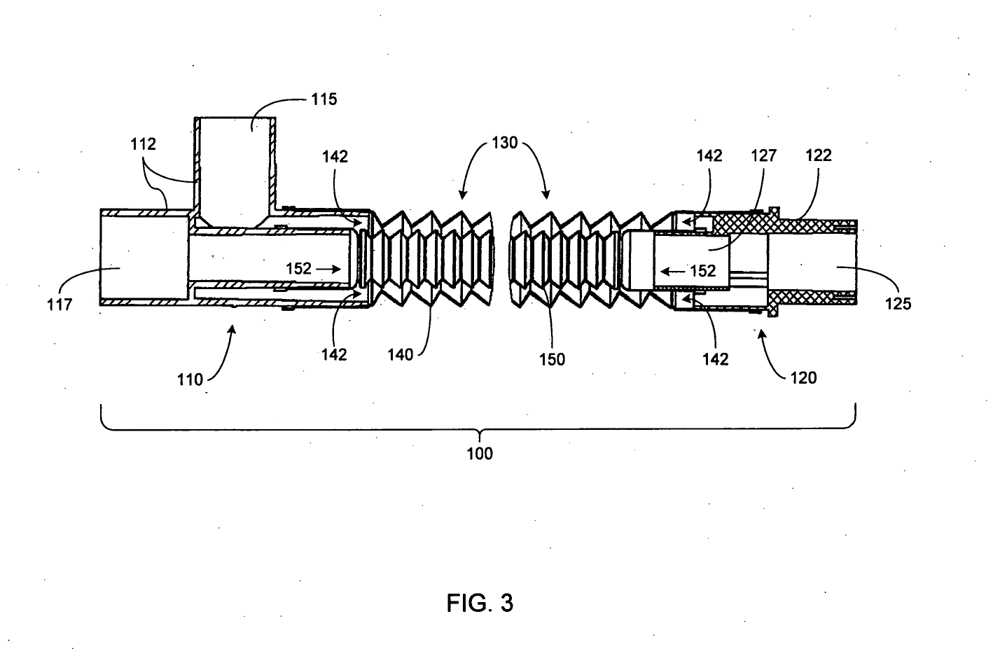

Referring to FIG. 3, substantially the same tube-in-tube breathing circuit assembly 100 is shown in cross-section. As can be seen in FIG. 3, the central region 130 of the assembly 100 includes an outer hose 140, and an inner hose 150 that extends loosely through an interior or passageway 142 of the outer hose 140. The inner hose 150 has an interior or passageway 152 extending therethrough. Because the inner hose 150 extends loosely through the interior or passageway 142 of the outer hose 140, the inner hose 150 to is permitted to extend and retract substantially independently from the outer hose 140.

Referring to the orientation of the assembly 100 as shown in FIG. 3, the first end connector 112 extends between and rigidly connects left end regions of the outer and inner hoses 140, 150, respectively, whereas the second end connector 122 extends between and rigidly connects right end regions of the outer and inner hoses 140, 150, respectively. The first end connector 112 provides tubular openings 115, 117 that communicate separately with the interiors or passageways 142, 152 of the outer and inner hoses 140, 150, respectively, at the left end regions of the outer and inner hoses 140, 150, respectively, whereas the second end connector 122 provides tubular openings 125, 127 that communicate separately with the interiors or passageways 142, 152 of the outer and inner hoses 140, 150, respectively.

The construction and arrangement of the end connectors 112, 122 is of a conventional nature, and is not a subject of importance to the present invention. As those skilled in the art will readily understand and appreciate, other types of end connectors (not shown) both existing and yet-to-be-devised can be employed by breathing circuit assemblies 100 that embody the present invention.

The outer and inner hoses 140, 150 both are corrugated in an accordion-like fashion that permits independent extension and retraction of the outer and inner hoses 140, 150. In one preferred embodiment of the invention, the inner hose 150 is “reverse set” as by annealing the inner hose 140 while it is axially compressed to a minimal axial length (as is described in greater detail in the referenced Hose Annealing Patents) so that, whenever the assembly 100 is extended, retracted or bent, the inner hose 150 will try to retract to its minimum axial length (and will therefore will try to remain taut and to maintain a relatively straight or linear configuration) when the assembly 100 is axially extended, retracted or bent.

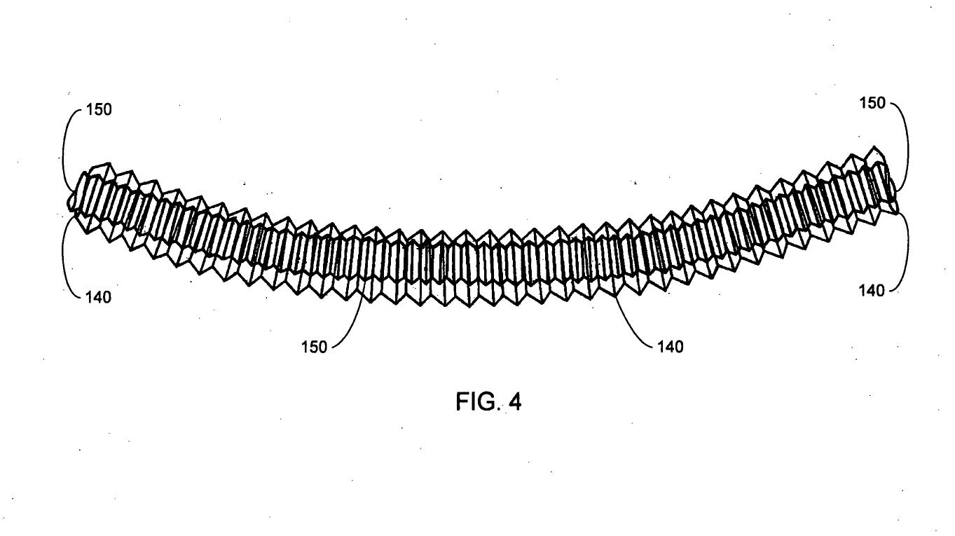

In contrast, the outer hose 140 is not stress relieved by being subjected to an annealing process such as is disclosed in the referenced Hose Annealing Patents—hence, when the central region 130 of the assembly 100 is extended in a manner such as is shown in FIG. 4, the tension in the inner hose 150 caused by its tendency to remain taut by retracting to a minimal length will cause the inner hose 150 to extend in as straight-line or linear a manner as possible within the outer hose 140. This tendency of the inner hose 150 to minimize its length or to remain taut causes the inner hose 150 to reside along the shortest radius of the passage 152 defined by of the interior surface of the wall of the curved or bent outer hose 140, as can be seen in FIG. 4 where, despite a downward pull of the force of gravity, the inner hose 150 can be seen to reside at an elevated position in close proximity to the uppermost part of the inner surface of the outer hose 140.

When the tube-in-tube breathing circuit assembly 100 is bent (for example, as is shown in FIG. 4), the tendency of the inner hose 150 to extend in as straight-line or linear shape as possible (as has just been explained) minimizes the resistance to flow presented by the inner hose 150 to breathing gas being ducted to a patient through the inner hose 150, and also causes the resistance to flow presented to exhaled breath being ducted away from the patient through the space between the outside surface of the inner hose 150 and the inside surface of the outer hose 140. This arrangement is much preferred over present day forms of breathing circuit assemblies which have inner hoses 150 that often tend to “bunch up” or extend in a tortuous spiral fashion through the passage 142 of the outer hose 140—an arrangement that significantly increases the flow resistance presented to exhaled breath that must find its way along a winding pathway between the inside surface of the outer hose 140, and the outside surface of the inner hose 150.

In some forms of preferred practice of the invention, a relatively large diameter outer hose 140 that has not been annealed to relieve stress in the manner disclosed in the referenced Hose Annealing Patents is used in combination with a relatively smaller diameter inner hose 150 that has been “reverse set” by being stress relieved while being axially compressed, as is disclosed in the referenced Hose Annealing Patents. By this arrangement, the inner hose 150 is always extensible, but always tries in a gentle way to minimize its length as it attempts to return to its “new normal, non-extended state.” However, characteristics of the outer hose 140 (which are stronger than characteristics of the inner hose 150) tend to dominate the behavior of the tube-in-tube breathing circuit assembly 100 that results when the gentle and easy-to-extend inner hose 150 is combined in the assembly 100 with the stronger outer hose 140.

In some forms of preferred practice, the outer hose 140 of the tube-in-tube breathing circuit assembly 100 is extensible, but it ordinarily has no noticeable tendency to retract if extended, or to straighten if bent. As such, the outer hose of the breathing circuit assembly can be said to be “normally collapsible” in that it can be extended, retracted and/or bent, and will tend to stay extended, retracted and/or bent to whatever extent it has been extended, retracted and/or bent.

Because the two hoses 140, 150 arranged one inside the other are rigidly coupled by the end connectors 112, 122 near opposite end regions, the gentle tendency of the inner hose 150 to always minimize its length tends to pull the end connectors 112, 122 toward each other—which, in turn, causes the end connectors 112, 122 to tend to minimize the length of the outer hose 140. However, the gentle squeeze-together force that is applied to the end connectors 112, 122 to opposite ends of the outer hose 140 by the “reverse set” memory of the inner hose 150 is too small in magnitude to alter the normal characteristics of the outer hose 140 which dominate the characteristics of the inner hose 150, and govern the behavior of the breathing circuit assembly 100 which tends to remain in whatever extended, retracted and/or bent configuration, form or attitude it may have assumed by being forcefully extended, retracted and/or bent.

In preferred practice, the outer hose 140 is formed from a semi-rigid polymer such as polypropylene, and has an accordion-like cross-section that is corrugated both interiorly and exteriorly. In preferred practice, the inner hose 150 is formed from a blend of polypropylene and metallocene in ratios that allow the inner hose to be softer than the outer hose. This allows the inner hose to obtain a better and more long lasting “reverse set” during an annealing process, and also gives the inner hose a relatively gentle tendency to retract.

Alternate thermoplastic materials that may be used during production of either of the outer or inner hoses 140, 150 include hytrel, metallocene, nylon, polyurethane, and blends of polypropylene and TPE materials—and other thermoplastic materials that may be known to those who are skilled in the art.

Whatever sizes of stretch hose are selected to form the outer and inner hoses 140, 150, respectively, the diameter of the outer hose 140 is preferably sufficiently larger than the diameter of the inner hose 150 so that the flow resistance that is presented by the assembly 100 to breathing gas and exhaled breath is minimized. Another consideration in selecting hose diameters is the amount of thermoplastic material that must be used to form the outer and inner hoses 140, 150, respectively, which needs to be minimized to hold down the cost of the assembly 100, and to ensure that the assembly 100 does not take on a clumsy feel or an inappropriately bulky appearance.

One breathing tube assembly 100 that has been found to work well is formed using an outer hose 140 of about 28 mm diameter, and an inner hose 150 of about 15 mm diameter. Another breathing tube assembly 100 that has been found to work well is formed using an outer hose 140 of about 22 mm diameter, and an inner hose 150 of about 10 mm diameter. The 28 to 15 ratio of about 1 to 1.87, or the 22 to 10 ratio of about 1 to 2.20 seems to define dimensions and a range of size ratios that work well with normal tube-in-tube breathing circuit assemblies 100 and with associated breathing equipment commonly in use today.

As will be apparent from the foregoing description, advantageous behavior is obtained by constructing a tube-in-tube breathing circuit assembly using an extensible-retractable inner hose that has a tendency to retract so as to remain relatively taut, and an outer hose that has a tendency to remain in such an extended, retracted and/or bent configuration as the assembly is put. In some instances, it may be able to provide a tube-in-tube breathing circuit assembly simply by selecting (prior to assembly) similar lengths of inner and outer hose that already have the desirably characteristics described herein.

However, as will also be apparent from the foregoing description, a preferable way in which the inner and outer hoses of a tube-in-tube breathing circuit assembly are obtained that have the desired type of extension and retraction characteristics and behaviors just described is 1) to anneal a length of inner hose stock of a desired diameter while axially compressing the hose length (as is described in the referenced Hose Annealing Patents) to give the length of inner hose a “reverse set,” and 2) to not anneal or otherwise stress-reduce a length outer hose stock of a desired diameter so the outer hose used in constructing a tube-in-tube breathing circuit assembly has no “reverse set,” and instead is selected from among corrugated stretch hoses that have a tendency to remain in whatever extended, retracted and/or bent configurations to which they are extended, retracted and/or bent.

Other techniques for providing inner and outer corrugated stretch hoses that have such desirable characteristics as are described herein may be known to, or may become known to, those who are skilled in the art—and can, therefore, be substituted or utilized as deemed appropriate to provide breathing circuit assemblies that have desired extension and retraction characteristics. For example, it may be possible to anneal, stress relieve, “reverse set” and/or otherwise treat similar lengths of inner and outer corrugated hose stock to lesser and greater degrees or in ways that differ—which might mean that features of the present invention can be attained by providing (in ways other than as described herein) an inner hose with a tendency to minimize its length, and a length of outer hose with a tendency to remain in such configurations to which it is extended, retracted and/or bent—all without departing from the spirit and scope of the present invention.

Although the invention has been described in its preferred form with a certain degree of particularity, it is understood that the present disclosure of the preferred form has been made only by way of example, and that numerous changes in the details of construction and the combination and arrangement of parts and techniques may be resorted to without departing from the spirit and scope of the invention as hereinafter claimed. It is intended to protect whatever features of patentable novelty that exist in the invention disclosed.

What is claimed is:

1. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors attached to, and communicating separately with the interior of nearby end regions of each of the inner and outer hoses, with the inner hose being reverse set by annealing the inner hose at a time while the inner hose is axially compressed thereby causing the inner hose to have a tendency to minimize its length so as to be taut regardless of an extent to which the assembly is forcefully extended, retracted or bent, and with the outer hose having a tendency to remain in whatever configuration to which the outer hose is forcefully extended, retracted or bent.

2. The tube-in-tube breathing circuit assembly of claim 1 in which the inner and outer hoses are formed from thermoplastic materials, and in which the outer hose has not been annealed.

3. The tube-in-tube breathing circuit assembly of claim 1 in which the inner and outer hoses are formed from thermoplastic materials, and in which the outer hose has no tendency to retract if extended, and no tendency to straighten if bent.

4. The tube-in-tube breathing circuit assembly of claim 1 in which the outer hose is formed from polypropylene, or polypropylene blended with another thermoplastic polymer.

5. The tube-in-tube breathing circuit assembly of claim 1 in which the inner hose is formed from polypropylene, or polypropylene blended with another thermoplastic polymer in a ratio that causes the inner hose to be softer than the outer hose.

6. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors being attached to, and communicating separately with the interior of nearby end regions of each of the inner and outer hoses, with the length of inner hose being reverse set, and with the length of outer hose having a tendency to retain a configuration to which the outer hose is caused to be extended, retracted or bent during use of the assembly.

7. The tube-in-tube breathing circuit assembly of claim 6 in which the inner and outer hoses are formed from thermoplastic materials.

8. The tube-in-tube breathing circuit assembly of claim 6 in which the inner and outer hoses are formed from thermoplastic materials, and in which the outer hose has no tendency to retract if extended, and no tendency to straighten if bent.

9. The tube-in-tube breathing circuit assembly of claim 6 in which the outer hose is formed from polypropylene, or polypropylene blended with another thermoplastic polymer.

10. The tube-in-tube breathing circuit assembly of claim 6 in which the inner hose is formed from polypropylene, or polypropylene blended with another thermoplastic polymer.

11. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors being attached to, and communicating separately with the interiors of nearby end regions of each of the inner and outer hoses, with the length of inner hose being reverse set to cause the length of inner hose to tend to remain taut regardless of an extent to which the inner hose may be extended during use of the assembly, and with the length of outer hose not being reverse set.

12. The tube-in-tube breathing circuit assembly of claim 11 in which the inner and outer hoses are formed from thermoplastic materials.

13. The tube-in-tube breathing circuit assembly of claim 11 in which the inner and outer hoses are formed from thermoplastic materials, and in which the outer hose has no tendency to retract if extended, and no tendency to straighten if bent.

14. The tube-in-tube breathing circuit assembly of claim 11 in which the outer hose is formed from polypropylene, or polypropylene blended with another thermoplastic polymer.

15. The tube-in-tube breathing circuit assembly of claim 11 in which the inner hose is formed from polypropylene, or polypropylene blended with another thermoplastic polymer.

16. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors being attached to, and communicating separately with the interiors of nearby end regions of each of the inner and outer hoses, with the length of inner hose having a tendency to remain taut regardless of an extent the assembly is extended, retracted or bent during use, and with the length of outer hose having a tendency to retain whatever configuration to which the length of outer hose is extended, retracted or bent during use of the assembly.

17. The tube-in-tube breathing circuit assembly of claim 16 in which the inner and outer hoses are formed from thermoplastic materials.

18. The tube-in-tube breathing circuit assembly of claim 16 in which the inner and outer hoses are formed from thermoplastic materials, and in which the outer hose has no tendency to retract if extended, and no tendency to straighten if bent.

19. The tube-in-tube breathing circuit assembly of claim 16 in which the outer hose is formed from polypropylene, or polypropylene blended with another thermoplastic polymer.

20. The tube-in-tube breathing circuit assembly of claim 16 in which the inner hose is formed from polypropylene, or polypropylene blended with another thermoplastic polymer.

21. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors being attached to, and communicating separately with the interiors of nearby end regions of each of the inner and outer hoses, with the length of inner hose having a tendency to retract to a minimal length when extended from said minimal length, and with the length of outer hose having a tendency to retain a configuration to which the outer hose is retracted, extended or bent during use of the assembly.

22. A flexible, stretchable tube-in-tube breathing circuit assembly formed from a length of corrugated, flexible, extensible-retractable inner hose of a first diameter extending loosely through a similar length of corrugated, flexible, extensible-retractable outer hose of a second, larger diameter, with the assembly also including first and second connectors each located near a different one of the opposite end regions of the assembly, with each of the first and second connectors attached to, and communicating separately with the interior of nearby end regions of each of the inner and outer hoses, with the length of inner hose having a relatively gentle tendency to minimize its length and to thereby remain taut regardless of an extent to which the assembly may be extended, retracted or bent during use, and with the length of outer hose having a dominant tendency to remain in whatever configuration to which the outer hose is forcefully extended, retracted or bent, thereby causing the assembly to have a tendency to remain in whatever configuration to which the assembly is forcefully extended, retracted or bent.
